(19) United States
(12) Patent Application Publication
(10) Pub. No.: US 2024/0030253 A1
(43) Pub. Date: Jan. 25, 2024

(54) IMAGING DEVICE AND ELECTRONIC APPARATUS

(71) Applicant: SONY SEMICONDUCTOR SOLUTIONS CORPORATION, Kanagawa (JP)

(72) Inventor: Tomohiko BABA, Kanagawa (JP)

(73) Assignee: SONY SEMICONDUCTOR SOLUTIONS CORPORATION, Kanagawa (JP)

(21) Appl. No.: 18/255,421

(22) PCT Filed: Dec. 1, 2021
(86) PCT No.: PCT/JP2021/044166

§ 371 Date: Jun. 1, 2023

(30) Foreign Application Priority Data
Dec. 9, 2020 (JP) 2020-204154

(51) Int. Cl. H01L 27/146

(52) U.S. Cl. CPC H01L 27/14627; H01L 27/14623; H01L 27/14625; A61B 90/20

(57) ABSTRACT

Provided are an imaging device capable of obtaining a high-quality image with high light utilization efficiency, and an electronic apparatus using the imaging device. An imaging device according to the present disclosure includes at least one pixel, in which the pixel includes a lens unit that condenses incident light, a phase modulation unit that modulates a phase of some light passed through the lens unit, a light-shielding unit including a pinhole that lets light of which phase is modulated in the phase modulation unit and light of which phase is not modulated pass through, and an imaging element that images light passed through the pinhole.

### TECHNICAL FIELD

Embodiments of the present disclosure relate to an imaging device and an electronic apparatus.

### BACKGROUND ART

There is known an image recognition system including an imaging device (sensor) having a plurality of pixels and a microlens array having a plurality of microlenses of the same size level as a unit pixel of the imaging device.

In the image recognition system, one or two pinholes are provided between each microlens and a corresponding sensor. Then, with respect to an optical axis of a microlens positioned at the center of the microlens array, the closer the microlens is to a peripheral-edge side, the larger inclination angle of the optical axis is. With this arrangement, a subject image can be recognized with a wide angle of view.

### CITATION LIST

Patent Document
Patent Document 1: Japanese Patent No. 5488928
Patent Document 2: Japanese Unexamined Patent Application No. 2007-520743

### SUMMARY OF THE INVENTION

### Problems to be Solved by the Invention

However, in the image recognition system, an area of incident light is limited by using pinholes, and therefore, light utilization efficiency is low. Furthermore, the closer to a peripheral edge of the microlens array, the more distorted ellipse a cross-section of a light flux passing through a circular pinhole becomes, and therefore, a light beam having a desired angle of view cannot be selected with a pinhole to reach a sensor array, and it is difficult to obtain high quality image quality.

The present disclosure provides an imaging device capable of obtaining a high-quality image with high light utilization efficiency, and an electronic apparatus using the imaging device.

### Solutions to Problems

An imaging device according to a first aspect of the present disclosure includes at least one pixel, in which the pixel
includes a lens unit that condenses incident light, a phase modulation unit that modulates a phase of some light passed through the lens unit, a light-shielding unit including a pinhole that lets light of which phase is modulated in the phase modulation unit and light of which phase is not modulated pass through, and an imaging element that images light passed through the pinhole.

In the imaging device according to the first aspect, the phase modulation unit may include a first part and a second part, and may be configured to generate a phase difference of ½ of a wavelength between light passed through the first part and light passed through the second part.

In the imaging device according to the first aspect, in the phase modulation unit, the first part and the second part may have substantially the same area.

The imaging device according to the first aspect may further include a first light guide unit that is disposed closer to a subject than the lens unit is and guides subject light to the lens unit.

The imaging device according to the first aspect may further include a second light guide unit that is disposed between the phase modulation unit and the light-shielding unit, and guides the light passed through the phase modulation unit to the light-shielding unit.

In the imaging device according to the first aspect, the light-shielding unit may include a first member including the pinhole and disposed in a direction intersecting a direction in which the light passed through the phase modulation unit propagates, and a second member extending from a peripheral edge portion of the first member in a direction of the phase modulation unit, and disposed on a side portion of the second light guide unit.

An imaging device according to a second aspect includes a plurality of pixels arranged in a matrix, in which each of the pixels includes a lens unit that condenses incident light, a light-shielding unit including a pinhole that lets at least a portion of light passed through the lens unit pass through, and an imaging element that images light passed through the pinhole, at least one pixel of the plurality of pixels includes a phase modulation unit that modulates a phase of the some light passed through the lens unit, and the light-shielding unit of the pixel including the phase modulation unit includes the pinhole that lets light of which phase is modulated in the phase modulation unit and light of which phase is not modulated pass through.

In the imaging device according to the second aspect, the phase modulation unit may include a first part and a second part, and may be configured to generate a phase difference of ½ of a wavelength between light passed through the first part and light passed through the second part.

In the imaging device according to the second aspect, in the phase modulation unit, the first part and the second part may have substantially the same area.

The imaging device according to the second aspect may further include a first light guide unit that is disposed closer to a subject than the lens unit is and guides subject light to the lens unit, and a second light guide unit that is disposed between the lens unit and the light-shielding unit, and guides a light beam from the lens unit to the light-shielding unit.

In the imaging device according to the second aspect, the light-shielding unit may, with a first member including the pinhole and disposed in a direction intersecting a direction in which the light passed through the phase modulation unit propagates, extend from a peripheral edge portion of the first member in a direction of the phase modulation unit, and be disposed on a side portion of the second light guide unit.

In the imaging device according to the second aspect, the plurality of pixels may be disposed in units of a pixel group including first to fourth pixels disposed in two rows and two columns adjacent to each other, the first to third pixels may include the phase modulation unit, and the fourth pixel may not include the phase modulation unit, and include a color filter disposed between the imaging element and the lens unit.

In the imaging device according to the second aspect, the plurality of pixels may be disposed in units of a pixel group including first to fourth pixels disposed in two rows and two columns adjacent to each other, the first to second pixels may include the phase modulation unit, and the third to fourth pixels may not include the phase modulation unit, and include different color filters disposed between the imaging element and the lens unit.

In the imaging device according to the second aspect, the plurality of pixels may be disposed in units of a pixel group including first to fourth pixels disposed in two rows and two columns adjacent to each other, the first pixel may include the phase modulation unit, and the second to fourth pixels may not include the phase modulation unit, and include color filters different from one another, the color filters being disposed between the imaging element and the lens unit.

In the imaging device according to the second aspect, the plurality of pixels may be disposed in units of a pixel group including first to fourth pixels disposed in two rows and two columns adjacent to each other, the first pixel may include the phase modulation unit, the second to third pixels may not include the phase modulation unit, and include first color filters of same color, the first color filters being disposed between the imaging element and the lens unit, and the fourth pixel may not include the phase modulation unit, and include a second color filter of a color different from the color of the first filters, the second color filter being disposed between the imaging element and the lens unit.

The imaging device according to the second aspect may further include an optical member that is disposed between the plurality of pixels and a subject, and condenses light from the subject on the plurality of pixels.

In the imaging device according to the second aspect. The optical member may be a convex lens.

In the imaging device according to the second aspect, the optical member may be a Fresnel lens.

In the imaging device according to the second aspect, the optical member may be a hologram.

An electronic apparatus according to a third aspect includes an imaging device, and a signal processing unit that performs signal processing on the basis of a pixel signal imaged in the imaging device, in which the imaging device includes a plurality of pixels arranged in a matrix, each of the pixels includes a lens unit that condenses incident light, a light-shielding unit including a pinhole that lets at least a portion of light passed through the lens group pass through, and an imaging element that images light passed through the pinhole, at least one pixel of the plurality of pixels includes a phase modulation unit that modulates a phase of the some light passed through the lens unit, and
the light-shielding unit of the pixel including the phase modulation unit includes the pinhole that lets light of which phase is modulated in the phase modulation unit and light of which phase is not modulated pass through.

### BRIEF DESCRIPTION OF DRAWINGS

FIG. 1 is a cross-sectional view illustrating a configuration of pixels in an imaging device according to a first embodiment.

FIG. 2 is a plan view illustrating phase modulation units of the pixels in the imaging device according to the first embodiment.

FIG. 3 is a diagram illustrating an effect of the imaging device according to the first embodiment.

FIG. 4 is a circuit diagram illustrating a configuration of the imaging device according to the first embodiment.

FIG. 5 is a plan view illustrating phase modulation units of pixels in an imaging device according to a modification of the first embodiment.

FIG. 6 is a diagram illustrating a pixel array of an imaging device according to a second embodiment.

FIG. 7 is a diagram illustrating a pixel array of an imaging device according to a first modification of the second embodiment.

FIG. 8 is a diagram illustrating a pixel array of an imaging device according to a second modification of the second embodiment.

FIG. 9 is a diagram illustrating a pixel array of an imaging device according to a third modification of the second embodiment.

FIG. 10 is a diagram illustrating an imaging device according to a third embodiment.

FIG. 11 is a cross-sectional view illustrating an example of an optical member of an imaging device according to a first modification of the third embodiment.

FIGS. 12A and 12B are a cross-sectional view and a top view, respectively, illustrating an example of the optical member of the imaging device according to the first modification of the third embodiment.

FIG. 13 is a diagram illustrating a configuration of an electronic apparatus according to a fourth embodiment.

FIG. 14 is a cross-sectional view illustrating a configuration of an imaging device according to a fifth embodiment.

FIG. 15 is a cross-sectional view illustrating a manufacturing process of an imaging device according to a sixth embodiment.

FIG. 16 is a cross-sectional view illustrating a manufacturing process of the imaging device according to the sixth embodiment.

FIG. 17 is a cross-sectional view illustrating a manufacturing process of the imaging device according to the sixth embodiment.

FIG. 18 is a cross-sectional view illustrating a manufacturing process of the imaging device according to the sixth embodiment.

FIG. 19 is a cross-sectional view illustrating a manufacturing process of the imaging device according to the sixth embodiment.

FIG. 20 is a cross-sectional view illustrating a manufacturing process of the imaging device according to the sixth embodiment.

FIG. 21 is a cross-sectional view illustrating a manufacturing process of the imaging device according to the sixth embodiment.

FIG. 22 is a cross-sectional view illustrating a manufacturing process of the imaging device according to the sixth embodiment.

FIG. 23 is a cross-sectional view illustrating a manufacturing process of the imaging device according to the sixth embodiment.

FIG. 24 is a cross-sectional view illustrating a manufacturing process of a hologram used in the third to fourth embodiments.

FIG. 25 is a cross-sectional view illustrating the manufacturing process of the hologram used in the third to fourth embodiments.

FIG. 26 is a cross-sectional view illustrating the manufacturing process of the hologram used in the third to fourth embodiments.

FIG. 27 is a cross-sectional view illustrating the manufacturing process of the hologram used in the third to fourth embodiments.

FIG. 28 is a cross-sectional view illustrating the manufacturing process of the hologram used in the third to fourth embodiments.

FIG. 29 is a cross-sectional view illustrating the manufacturing process of the hologram used in the third to fourth embodiments.

FIG. 30 is a cross-sectional view illustrating the manufacturing process of the hologram used in the third to fourth embodiments.

FIG. 31 is a cross-sectional view illustrating the manufacturing process of the hologram used in the third to fourth embodiments.

FIG. 32 is a cross-sectional view illustrating the manufacturing process of the hologram used in the third to fourth embodiments.

FIG. 33 is a cross-sectional view illustrating the manufacturing process of the hologram used in the third to fourth embodiments.

FIG. 34 is a cross-sectional view illustrating the manufacturing process of the hologram used in the third to fourth embodiments.

FIG. 35 is a cross-sectional view illustrating the manufacturing process of the hologram used in the third to fourth embodiments.

FIG. 36 is a cross-sectional view illustrating the manufacturing process of the hologram used in the third to fourth embodiments.

FIG. 37 is a cross-sectional view illustrating the manufacturing process of the hologram used in the third to fourth embodiments.

FIG. 38 is a cross-sectional view illustrating the manufacturing process of the hologram used in the third to fourth embodiments.

### MODES FOR CARRYING OUT THE INVENTION

Embodiments of the present disclosure are described with reference to the drawings. Although components of an imaging device and electronic apparatus are mainly described hereinafter, the imaging device and the electronic apparatus may have components and functions that are not illustrated or described. The following description does not exclude components and functions that are not illustrated or described.

Furthermore, the drawings referred to in the following description are drawings for illustrating the embodiments of the present disclosure and promoting understanding thereof, and shapes, dimensions, ratios and the like in the drawings might be different from actual ones for the sake of clarity.

### First Embodiment

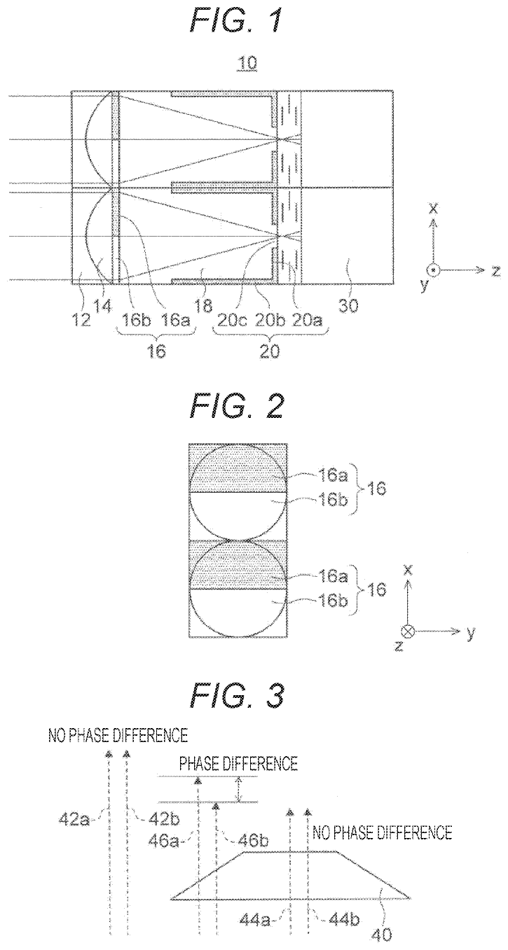

An imaging device according to a first embodiment will be described with reference to FIGS. 1 to 5. The imaging device according to the first embodiment is an image sensor, and includes a pixel array including a plurality of pixels arranged in a matrix. FIG. 1 is a cross-sectional view of pixels arranged in one direction. In FIG. 1, a plurality of (for example, two) pixels 10 is arranged in an x direction, but a plurality of pixels (not illustrated) may be arranged in a y direction. That is, in a case where m and n are positive integers, in the pixel array, the pixels 10 may be arranged in a matrix of m rows x n columns. Note that a z direction is a direction in which light from a subject (not illustrated) propagates.

Each of the pixels 10 includes a light guide unit 12, a lens unit 14, a phase modulation unit 16, a light guide unit 18, a light-shielding unit 20, and an imaging element 30.

The light guide unit 12 is disposed between a subject (not illustrated) and the imaging element 30, and propagates light from the subject to the lens unit 14. The light guide unit 12 includes a material through which visible light is transmitted, and for example, a material having a refractive index of 1.55 for sodium D-lines (hereinafter, also simply referred to as the D-lines) is used.

The lens unit 14 is disposed between the light guide unit 12 and the imaging element 30, and propagates the light propagated through the light guide unit 12 to the phase modulation unit 16. The lens unit 14 includes, for example, a convex microlens, and the microlens has, for example, a refractive index of 1.9 for the D-lines.

The phase modulation unit 16 is disposed between the lens unit 14 and the imaging element 30, splits the light propagated from the lens unit 14 into two beams of light having different phases (for example, having a phase difference of a half-wave length of a visible light wavelength X), and propagates the light beams to the light guide unit 18. For example, as illustrated in FIG. 2, the phase modulation unit 16 is divided into two parts 16a and 16b. For example, the part 16a includes a transparent material having a refractive index of 1.9 for the D-lines, the part 16b includes a transparent material having a refractive index of, for example, 1.55 for the D-lines, and transparent materials having a thickness (length in the z direction) of, for example, 0.7857 m are used. In FIG. 2, an area of the part 16a and an area of the part 16b when viewed from the subject are substantially equal. That is, a difference between the area of the part 16a and the area of the part 16b is within a manufacturing tolerance.

With this configuration, a phase difference of a ½ wavelength occurs between the light transmitted through the part 16a and the light transmitted through the part 16b. Note that FIG. 2 is. A plan view of the phase modulation unit 16 viewed from the subject. In FIG. 2, a circular solid line indicates an area of a light flux that has passed through the lens unit 14.

The light guide unit 18 is disposed between the phase modulation unit 16 and the imaging element 30, and propagates the light from the phase modulation unit 16 to the light-shielding unit 20. Similarly to the light guide unit 12, the light guide unit 18 includes a material through which visible light is transmitted, and for example, a material having a refractive index of 1.55 for the D-lines is used.

The light-shielding unit 20 is disposed between the light guide unit 18 and the imaging element 30, and includes a part 20a provided on a surface of the light guide unit 18, the surface facing the imaging element 30, and a part 20b provided on a portion of a side surface (a surface parallel to the z direction) of the light guide unit 18. The part 20a is disposed at a position away from the lens unit 14 by a substantial focal length of the microlens, and is provided with a pinhole 20c at the center. Here, the “substantial focal length” means a focal length within a range of a manufacturing tolerance with the microlens. Note that the focal length of the microlens is preferably longer than 0.0003 mm and shorter than 3 mm. Furthermore, a diameter of the pinhole is preferably larger than 0.1 m and smaller than 2 km.

The part 20b is provided on the side surface of the light guide unit 18, the side being close to the imaging element 30, and prevents the light from propagating from the side surface of the light guide unit 18 to another pixel. That is, the light-shielding unit 20 propagates the light beams, which are condensed by the lens unit 14 via the phase modulation unit 16 and the light guide unit 18, to the imaging element 30 through the pinhole 20c, and prevents, with the part 20b, the light from propagating to an adjacent pixel to cause crosstalk. That is, the parts 20a and 20b include a material that absorbs visible light.

The imaging element 30 includes a charge-coupled device (CCD) or a CMOS image sensor element.

In each pixel 10, an optical axis of an optical system including the light guide unit 12, the lens unit 14, the phase modulation unit 16, the light guide unit 18, and the light-shielding unit 20 passes through substantially the center of the imaging element 30.

In the present embodiment, the phase modulation unit 16 is provided, in which the phase modulation unit 16 splits, with the part 16a and the part 16b, the light passing through the phase modulation unit 16 into two beams of light having a phase difference of a half-wave length. With this arrangement, a nearly transparent subject, such as a cell, can be observed more easily. This will be described below with reference to FIG. 3. For example. As illustrated in FIG. 3, a case is considered where a subject 40 has a trapezoidal cross-section and is nearly transparent, and light is incident on the subject 40 from an opposite side of an imaging element, that is, where the light is incident on the subject 40 from a lower side of the trapezoid and is emitted to a pixel from an upper side of the trapezoid. It is assumed that a pair of two light beams is photoelectrically converted by the imaging element of the same pixel. One of the two light beams represents light passing through the center of an area of the part 16a of the phase modulation unit 16, and another of the two light beams represents light passing through the center of an area of the part 16b of the phase modulation unit 16.

Because light beams 42a and 42b illustrated in FIG. 3 do not pass through the subject 40, no phase difference occurs between the two light beams 42a and 42b. Therefore, a phase difference between the light beam 42a passing through the part 16a of the phase modulation unit 16 and the light beam 42b passing through the part 16b of the phase modulation unit 16 is canceled by the corresponding imaging element, and thus an image the same as an image obtained in a case where there is no phase difference can be obtained. Because light beams 44a and 44b illustrated in FIG. 3 are transmitted through the lower side and upper side of the trapezoidal shape of the subject 40, no phase difference occurs between the transmitted light beams 44a and 44b, and, similarly to a case of the light beams 42a and 42b, a phase difference is canceled by the corresponding imaging element, and thus an image the same as an image obtained in a case where there is no phase difference can be obtained. Light beams 46a and 46b illustrated in FIG. 3 pass through different positions on an oblique side of the subject 40, and therefore have a phase difference. Therefore, a phase of the light beam passing through the center of the area of the part 16a of the phase modulation unit 16 and a phase of the light beam passing through the center of the area of the part 16b of the phase modulation unit 16 are different, and thus the phase difference is not canceled by the corresponding imaging element, and an image having a phase difference is obtained. That is, an image having differential interference contrast can be obtained, and thus a high-quality image can be obtained.

Note that, in the first embodiment, the part 16a and the part 16b are separately disposed in the x direction in the phase modulation unit 16, and therefore, contrast of the image is generated along the x direction, and the obtained image is like a three-dimensional image viewed from the x direction.

In the present embodiment, the light is condensed into the pinhole 20c of the light-shielding unit 20 by using the lens unit 14 including the microlens, and therefore light utilization efficiency can be increased. Furthermore, because a portion of the side surface of the light guide unit 18 is covered with the part 20b of the light-shielding unit 20, crosstalk can be reduced, and light utilization efficiency can be further increased.

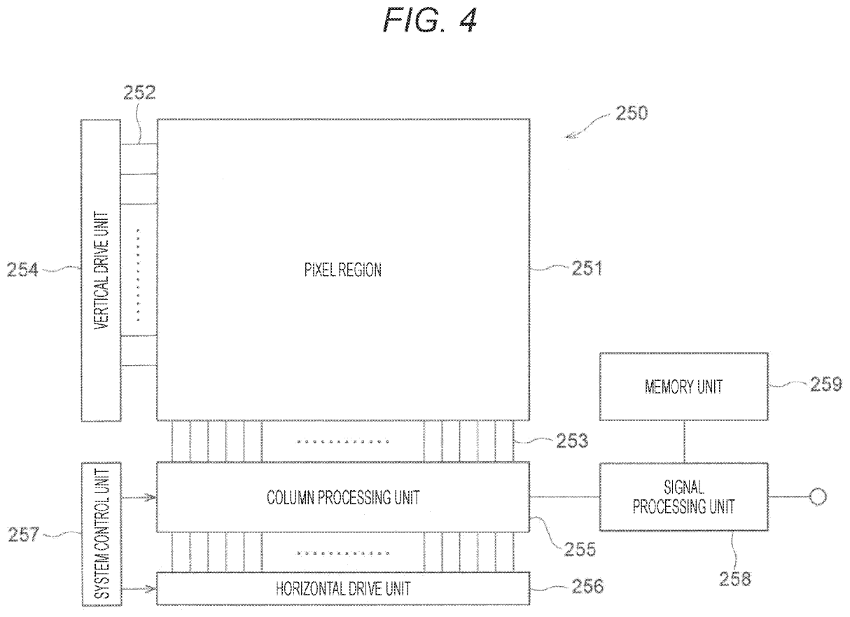

FIG. 4 illustrates an overall configuration of the imaging device according to the present embodiment. An imaging device 250 according to the present embodiment includes a pixel region 251, pixel drive lines 252, vertical signal lines 253, a vertical drive unit 254, a column processing unit 255, a horizontal drive unit 256, a system control unit 257, a signal processing unit 258, and a memory unit 259. These are formed on a semiconductor substrate (chip) such as a silicon substrate (not illustrated). Note that the pixel region 251 may be formed on a sensor chip including a first semiconductor substrate, the pixel drive line 252, the vertical signal line 253, the vertical drive unit 254, the column processing unit 255, the horizontal drive unit 256, the system control unit 257, the signal processing unit 258, and the memory unit 259 may be formed on a circuit chip including a second semiconductor substrate, and these chips may be bonded together.

The pixel region 251 is a pixel array in which the pixels 10 described in FIG. 1 are two-dimensionally arranged, and converts an optical signal into an electric signal to perform imaging. In the pixel region 251, with respect to pixels, a pixel drive line 252 is provided every two rows, and a vertical signal line 253 is provided every two columns.

The vertical drive unit 254 includes a shift register, an address decoder, or the like, and supplies drive signals to the pixel drive lines 252 so that pixel signals corresponding to charges accumulated in the respective imaging elements of the pixel region 251 are read row by row from the top, in order of an odd column and an even column.

The column processing unit 255 includes a signal processing circuit for each two columns of the pixel region 251. Each signal processing circuit of the column processing unit 255 performs signal processing, such as A/D conversion processing or correlated double sampling (CDS) processing, on the pixel signals read from the corresponding pixels and supplied through the vertical signal lines 253. The column processing unit 255 temporarily holds the pixel signals subjected to the signal processing.

The horizontal drive unit 256 includes a shift register, an address decoder, or the like, and sequentially selects signal processing circuits of the column processing unit 255. With this arrangement, the pixel signals subjected to the signal processing by the respective signal processing circuits of the column processing unit 255 are sequentially output to the signal processing unit 258.

The system control unit 257 includes, for example, a timing generator that generates various timing signals, and controls the vertical drive unit 254, the column processing unit 255, and the horizontal drive unit 256 on the basis of the various timing signals generated by the timing generator.

The signal processing unit 258 performs various kinds of signal processing on the pixel signals output from the column processing unit. At this time, the signal processing unit 258 stores, in the memory unit 259, an intermediate result of the signal processing, or the like, as necessary, and refers to the result at a necessary timing. The signal processing unit 258 outputs the pixel signals subjected to the signal processing.

The memory unit 259 includes a dynamic random access memory (DRAM), a static random access memory (SRAM), or the like.

As described above, according to the present embodiment, it is possible to provide an imaging device capable of obtaining a high-quality image with high light utilization efficiency.

(Modifications)

In the first embodiment, contrast is generated in the obtained image in the x direction, and therefore, it is possible to see a difference in thickness of the subject in the x direction. However, it is difficult to obtain a difference in thickness of the subject in the y direction.

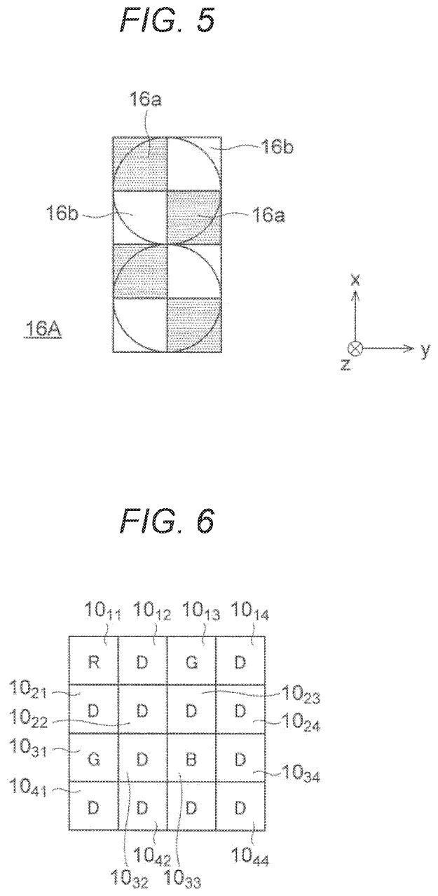

Therefore, it is possible to see a difference in thicknesses of the subject in the x direction and in the y direction in the imaging device according to the first embodiment, by using, as a modification of the first embodiment, a phase modulation unit 16A illustrated in FIG. 5 instead of the phase modulation unit 16 illustrated in FIG. 2. The phase modulation unit 16A has a configuration in which parts 16a are disposed in two regions positioned on one diagonal line among regions divided into two in the x direction and divided into two in the y direction, and a parts 16b are disposed in two regions positioned on another diagonal line. In FIG. 5 also, areas of the parts 16a and the parts 16b are substantially equal.

As an example, in a case where a specific wavelength (for example, 940 nm) is incident on all the pixels of the imaging device, a ½-wave phase plate of the above-described specific wavelength can be used as a part 16a of the phase modulation unit 16A of each pixel.

Similarly to the first embodiment, this modification can also provide an imaging device capable of obtaining a high-quality image with high light utilization efficiency.

### Second Embodiment

An imaging device according to a second embodiment will be described with reference to FIG. 6. While the first embodiment is an imaging device that can obtain a monochrome image, the second embodiment is an imaging device that can obtain a color image.

FIG. 6 is a diagram illustrating a pixel array of the imaging device according to the second embodiment. Pixels 10₁₁ to 10₄₄ disposed in four rows and four columns are divided into first to fourth pixel groups, each pixel group including four pixels disposed in two rows and two columns as one unit. For example, a first pixel group includes pixels 10₁₁, 10₁₂, 10₂₁, and 10₂₂, a second pixel group includes pixels 10₁₃, 10₁₄, 10₂₃, and 10₂₄, a third pixel group includes pixels 10₃₁, 10₃₂, 10₄₁, and 10₄₂, and a fourth pixel group includes pixels 10₃₃, 10₃₄, 10₄₃, and 10₄₄.

In the second embodiment, one pixel (for example, the pixel 10₁₁) of the four pixels 10₁₁, 10₁₂, 10₂₁, and 10₂₂ in the first pixel group is a red pixel (hereinafter, a red pixel is also referred to as an R pixel), and the other pixels are differential interference pixels (hereinafter, a differential interference pixel is also referred to as a D pixel).

One pixel (for example, the pixel 10₁₃) of the four pixels 10₁₃, 10₁₄, 10₂₃, and 10₂₄ in the second pixel group is a green pixel (hereinafter, a green pixel is also referred to as a G pixel), and the other pixels are D pixels.

One pixel (for example, the pixel 10₃₁) of the four pixels 10₃₁, 10₃₂, 10₄₁, and 10₄₂ in the third pixel group is a G pixel, and the other pixels are D pixels.

One pixel (for example, the pixel 10₃₃) of the four pixels 10₃₃, 10₃₄, 10₄₃, and 10₄₄ in the fourth pixel group is a blue pixel (hereinafter, a blue pixel is also referred to as a B pixel), and the other pixels are D pixels.

The D pixel has the same configuration as a pixel 10 illustrated in FIG. 1, and a phase modulation unit 16 illustrated in FIG. 2 or a phase modulation unit 16A illustrated in FIG. 5 is used as a phase modulation unit. Note that, in the second embodiment, each D pixel in the first pixel group uses a half-wave phase plate of R color as the phase modulation unit 16A, each D pixel in the second pixel group uses a half-wave phase plate of G color as the phase modulation unit 16A, each D pixel in the third pixel group uses a half-wave phase plate of G color as the phase modulation unit 16A, and each D pixel in the fourth pixel group uses a half-wave phase plate of B color as the phase modulation unit 16A.

The R pixel has a configuration in which the phase modulation unit 16 of the pixel 10 illustrated in FIG. 1 is replaced by a red filter, the G pixel has a configuration in which the phase modulation unit 16 of the pixel 10 illustrated in FIG. 1 is replaced by a green filter, and the B pixel has a configuration in which the phase modulation unit 16 of the pixel 10 illustrated in FIG. 1 is replaced by a B-color filter. With such a configuration, the first to fourth pixel groups represent a Bayer pattern when regarded as one unit of color reproduction, and a color image can be obtained.

Furthermore, one color pixel and the other three D pixels are provided in each pixel group, and each of these three D pixels includes a half-wave plate of the color of the color pixel, and therefore, an image in which the above-described color is enhanced can be obtained.

The second embodiment can also obtain a high-quality color image with high light utilization efficiency.

### First Modification

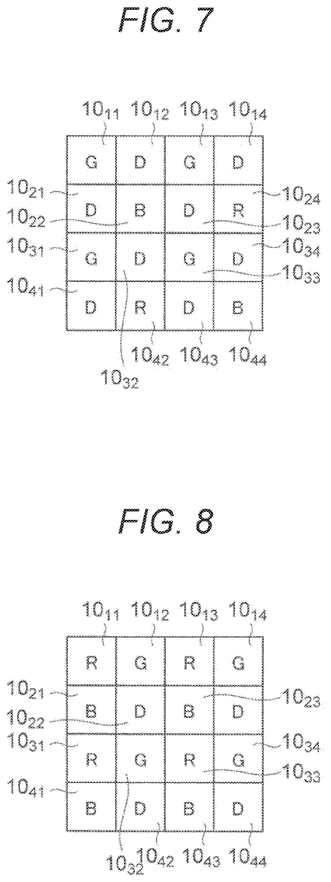

An imaging device according to a first modification of the second embodiment will be described with reference to FIG. 7. FIG. 7 is a diagram illustrating a pixel array of the imaging device according to the first modification of the second embodiment. Similarly to the second embodiment, pixels 10₁₁ to 10₄₄ disposed in four rows and four columns are divided into first to fourth pixel groups, each pixel group including four pixels disposed in two rows and two columns as one unit. For example, a first pixel group includes pixels 10₁₁, 10₁₂, 10₂₁, and 10₂₂, a second pixel group includes pixels 10₁₃, 10₁₄, 10₂₃, and 10₂₄, a third pixel group includes pixels 10₃₁, 10₃₂, 10₄₁, and 10₄₂, and a fourth pixel group includes pixels 10₃₃, 10₃₄, 10₄₃, and 10₄₄.

In the first modification of the second embodiment, one pixel (for example, the pixel 10₁₁) of the four pixels 10₁₁, 10₁₂, 10₂₁, and 10₂₂ in the first pixel group is a G pixel, another one pixel (for example, the pixel 10₂₂) is a B pixel, and the other two pixels are D pixels. Each of these D pixels uses a half-wave phase plate of G color as a phase modulation unit 16A.

One pixel (for example, the pixel 10₁₃) of the four pixels 10₁₃, 10₁₄, 10₂₃, and 10₂₄ in the second pixel group is a G pixel, another one pixel (for example, the pixel 10₂₄) is an R pixel, and the other two pixels are D pixels. Each of these D pixels uses a half-wave phase plate of G color as a phase modulation unit 16A.

One pixel (for example, the pixel 10₃₁) of the four pixels 10₃₁, 10₃₂, 10₄₁, and 10₄₂ in the third pixel group is a G pixel, another one pixel (for example, the pixel 10₄₂) is an R pixel, and the other two pixels are D pixels. Each of these D pixels uses a half-wave phase plate of G color as a phase modulation unit 16A.

One pixel (for example, the pixel 10₃₃) of the four pixels 10₃₃, 10₃₄, 10₄₃, and 10₄₄ in the fourth pixel group is a G pixel, another one pixel (for example, the pixel 10₄₄) is a B pixel, and the other two pixels are D pixels. Each of these D pixels uses a half-wave phase plate of B color as a phase modulation unit 16A.

In the first modification, each D pixel in the first to fourth pixel groups uses a half-wave phase plate of B color as a phase modulation unit 16A, and therefore, an image in which the B color is enhanced can be obtained.

With such a configuration, the first modification of the second embodiment can also obtain a high-quality color image with high light utilization efficiency.

### Second Modification

An imaging device according to a second modification of the second embodiment will be described with reference to FIG. 8. FIG. 8 is a diagram illustrating a pixel array of the imaging device according to the second modification of the second embodiment. Similarly to the second embodiment, pixels 10₁₁ to 10₄₄ disposed in four rows and four columns are divided into first to fourth pixel groups, each pixel group including four pixels disposed in two rows and two columns as one unit. For example, a first pixel group includes pixels 10₁₁, 10₁₂, 10₂₁, and 10₂₂, a second pixel group includes pixels 10₁₃, 10₁₄, 10₂₃, and 10₂₄, a third pixel group includes pixels 10₃₁, 10₃₂, 10₄₁, and 10₄₂, and a fourth pixel group includes pixels 10₃₃, 10₃₄, 10₄₃, and 10₄₄.

In the second modification of the second embodiment, one pixel (for example, the pixel 10₁₁) of the four pixels 10₁₁, 10₁₂, 10₂₁, and 10₂₂ in the first pixel group is an R pixel, another one pixel (for example, the pixel 10₁₂) is a G pixel, still another one pixel (for example, the pixel 10₂₁) is a B pixel, and the other one pixel (for example, the pixel 10₂₂) is a D pixel. The D pixel uses a half-wave phase plate of G color as a phase modulation unit 16A.

One pixel (for example, the pixel 10₁₃) of the four pixels 10₁₃, 10₁₄, 10₂₃, and 10₂₄ in the second pixel group is an R pixel, another one pixel (for example, the pixel 10₁₄) is a G pixel, still another one pixel (for example, the pixel 10₂₃) is a B pixel, and the other one pixel (for example, the pixel 10₂₄) is a D pixel. The D pixel uses a half-wave phase plate of G color as a phase modulation unit 16A.

One pixel (for example, the pixel 10₃₁) of the four pixels 10₃₁, 10₃₂, 10₄₁, and 10₄₂ in the third pixel group is an R pixel, another one pixel (for example, the pixel 10₃₂) is a G pixel, still another one pixel (for example, the pixel 10₄₁) is a B pixel, and the other one pixel (for example, the pixel 10₄₂) is a D pixel. The D pixel uses a half-wave phase plate of G color as a phase modulation unit 16A.

One pixel (for example, the pixel 10₃₃) of the four pixels 10₃₃, 10₃₄, 10₄₃, and 10₄₄ in the fourth pixel group is an R pixel, another one pixel (for example, the pixel 10₃₄) is a G pixel, still another one pixel (for example, the pixel 10₄₃) is a B pixel, and the other one pixel (for example, the pixel 10₄₄) is a D pixel. The D pixel uses a half-wave phase plate of G color as a phase modulation unit 16A.

That is, in the second modification, each of the first to fourth pixel groups has pixels in an identical array.

In the second modification, each D pixel in the first to fourth pixel groups uses a half-wave phase plate of B color as a phase modulation unit 16A, and therefore, an image in which the B color is enhanced can be obtained.

With such a configuration, the second modification of the second embodiment can also obtain a high-quality color image with high light utilization efficiency.

### Third Modification

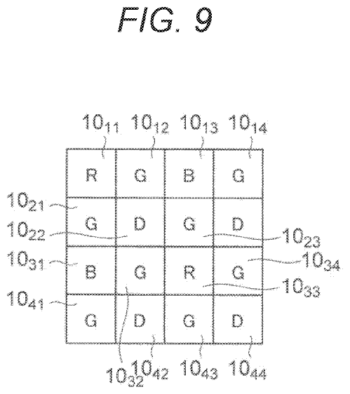

An imaging device according to a third modification of the second embodiment will be described with reference to FIG. 9. FIG. 9 is a diagram illustrating a pixel array of the imaging device according to the third modification of the second embodiment. Similarly to the second embodiment, pixels 10₁₁ to 10₄₄ disposed in four rows and four columns are divided into first to fourth pixel groups, each pixel group including four pixels disposed in two rows and two columns as one unit. For example, a first pixel group includes pixels 10₁₁, 10₁₂, 10₂₁, and 10₂₂, a second pixel group includes pixels 10₁₃, 10₁₄, 10₂₃, and 10₂₄, a third pixel group includes pixels 10₃₁, 10₃₂, 10₄₁, and 10₄₂, and a fourth pixel group includes pixels 10₃₃, 10₃₄, 10₄₃, and 10₄₄.

In the third modification of the second embodiment, one pixel (for example, the pixel 10₁₁) of the four pixels 10₁₁, 10₁₂, 10₂₁, and 10₂₂ in the first pixel group is an R pixel, other two pixels (for example, the pixels 10₁₂ and 10₂₁) are G pixels, and the other one pixel (for example, the pixel 10₂₂) is a D pixel. The D pixel uses a half-wave phase plate of G color as a phase modulation unit 16A.

One pixel (for example, the pixel 10₁₃) of the four pixels 10₁₃, 10₁₄, 10₂₃, and 10₂₄ in the second pixel group is a B pixel, other two pixels (for example, the pixels 10₁₄ and 10₂₃) are G pixels, and the other one pixel (for example, the pixel 10₂₄) is a D pixel. The D pixel uses a half-wave phase plate of G color as a phase modulation unit 16A.

One pixel (for example, the pixel 10₃₁) of the four pixels 10₃₁, 10₃₂, 10₄₁, and 10₄₂ in the third pixel group is a B pixel, other two pixels (for example, the pixels 10₃₂ and 10₄₁) are G pixels, and the other one pixel (for example, the pixel 10₄₂) is a D pixel. The D pixel uses a half-wave phase plate of G color as a phase modulation unit 16A.

One pixel (for example, the pixel 10₃₃) of the four pixels 10₃₃, 10₃₄, 10₄₃, and 10₄₄ in the fourth pixel group is an R pixel, other two pixels (for example, the pixels 10₃₄ and 10₄₃) are G pixels, and the other one pixel (for example, the pixel 10₄₄) is a D pixel. The D pixel uses a half-wave phase plate of G color as a phase modulation unit 16A.

In the third modification, each D pixel in the first to fourth pixel groups uses a half-wave phase plate of B color as a phase modulation unit 16A, and therefore, an image in which the B color is enhanced can be obtained.

With such a configuration, the third modification of the second embodiment can also obtain a high-quality color image with high light utilization efficiency.

### Third Embodiment

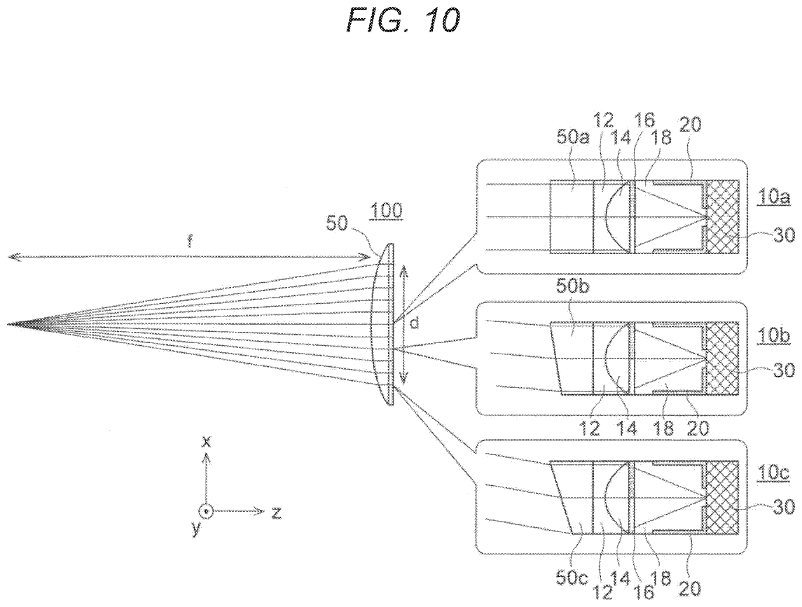

FIG. 10 illustrates a configuration of an imaging device according to a third embodiment. FIG. 10 is a cross-sectional view of the imaging device according to the third embodiment. The imaging device 100 according to the third embodiment has a configuration in which an optical member (for example, a convex lens) 50 is further provided on a pixel array in which a plurality of pixels 10 is arranged in a matrix in the imaging devices according to the first embodiment and second embodiment, and the modifications thereof.

In FIG. 10, three pixels 10a, 10b, and 10c in the pixel array are illustrated. The pixel 10a is a pixel corresponding to the center of an image obtained by the imaging device, and receives a light beam passing through a region 50a at the center of a convex lens 50. The light beam that has passed through the region 50a propagates to the light guide unit 12 of the pixel 10a.

The pixel 10c is a pixel corresponding to a vicinity of an edge of the image obtained by the imaging device, and receives a light beam passing through a region 50c in vicinity of an edge of the convex lens 50. The light beam that has passed through the region 50c propagates to the light guide unit 12 of the pixel 10c.

The pixel 10b is a pixel corresponding to a region between the center and vicinity of the edge of the image obtained by the imaging device, and receives a light beam passing through a region 50b between the region 50a at the center of the convex lens 50 and a region 50c in vicinity of the edge of the convex lens 50. The light beam that has passed through the region 50b propagates to the light guide unit 12 of the pixel 10b.

In the imaging device 100 according to the third embodiment, for example, an array pitch between the imaging elements 30 is 5 m, a diagonal length d of an effective diameter of the imaging device 100 is 3.3 mm, and a focal length f of the convex lens 50 is 10 mm.

In this case, when a subject (not illustrated) at a position 5 mm from the convex lens 50 is viewed, a diagonal length of the image is reduced from 3.3 mm to 1.65 mm, and a pixel pitch (resolution in a broad sense) is reduced from 5 m to 2.5 m. That is, the pixel pitch is reduced by ½.

Furthermore, when a subject (not illustrated) at a position 9 mm from the convex lens 50 is viewed, the diagonal length of the image is reduced from 3.3 mm to 0.33 mm, and the resolution is reduced from 5 m to 0.5 m. That is, the pixel pitch is reduced by 1/10.

Furthermore, when a subject (not illustrated) at a position 9.9 mm from the convex lens 50 is viewed, the diagonal length of the image is reduced from 3.3 mm to 0.033 mm, and the resolution is reduced from 5 m to 0.05 m. That is, the pixel pitch is reduced by 1/100.

Moreover, when a subject (not illustrated) at a position 9.95 mm from the convex lens 50 is viewed, the diagonal length of the image is reduced from 3.3 mm to 0.0066 mm, and the resolution is reduced from 5 m to 0.01 am. That is, the pixel pitch is reduced by 1/500.

As the above description illustrates, the imaging device according to the present embodiment is a microscope having functions equivalent to functions of a microscope, and not using an objective lens.

Note that the focal length of the convex lens 50 is preferably longer than 0.1 mm and shorter than 1000 mm.

Furthermore, because each pixel in the imaging device 100 according to the present embodiment is provided with the phase modulation unit 16, similarly to the imaging device according to the first embodiment, a nearly transparent subject, such as a cell, can be observed, and an image obtained has high image quality.

Furthermore, if the imaging device according to the second embodiment and the modifications thereof are used as the imaging device according to the present embodiment, it is possible to obtain an image in which a nearly transparent subject is subjected to color reproduction and color enhancement.

Note that the convex lens 50 usually includes a glass material. However, a plastic lens including a plastic material may be used. In this case, the convex lens 50 can be formed on the pixel array by using a replica process.

As described above, according to the third embodiment, similarly to the first embodiment, it is possible to provide an imaging device capable of obtaining a high-quality image with high light utilization efficiency.

### First Modification

An imaging device according to a first modification of the third embodiment will be described with reference to FIG. 11. The imaging device according to the first modification has a configuration of the imaging device 100 according to the third embodiment, in which the optical member 50 is replaced by a Fresnel lens 52 illustrated in FIG. 11. FIG. 11 is a cross-sectional view taken along a plane, including an optical axis, of the Fresnel lens 52.

In the first modification, similarly to the third embodiment, a nearly transparent subject, such as a cell, can be observed, and an image obtained has high image quality. Furthermore, if the imaging device according to the second embodiment and the modifications thereof are used as the imaging device according to the present modification, it is possible to obtain an image in which a nearly transparent subject is subjected to color reproduction and color enhancement.

As described above, according to the present modification, it is possible to provide an imaging device capable of obtaining a high-quality image with high light utilization efficiency.

### Second Modification

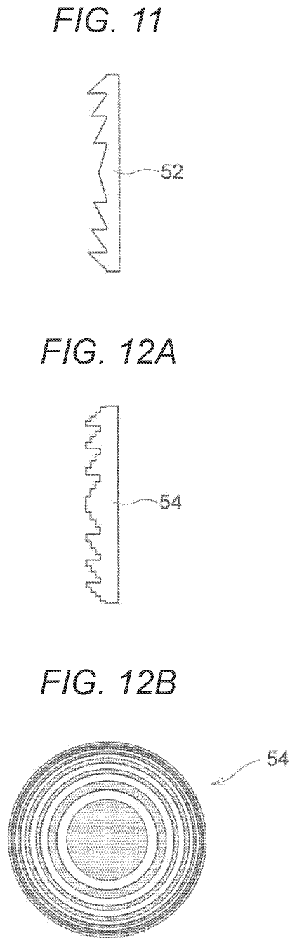

An imaging device according to a second modification of the third embodiment will be described with reference to FIGS. 12A and 12B. The imaging device according to the second modification has a configuration of the imaging device 100 according to the third embodiment, in which the optical member 50 is replaced by a hologram 54 illustrated in FIGS. 12A and 12B. FIG. 12A is a cross-sectional view taken along a plane including an optical axis of the hologram 54, and FIG. 12B is a top view of the hologram 54. Unlike a lens or the like utilizing a refraction phenomenon, the hologram 54 utilizes a diffraction phenomenon.

In the first modification, similarly to the third embodiment, a nearly transparent subject, such as a cell, can be observed, and an image obtained has high image quality. Furthermore, if the imaging device according to the second embodiment and the modifications thereof are used as the imaging device according to the present modification, it is possible to obtain an image in which a nearly transparent subject is subjected to color reproduction and color enhancement.

As described above, according to the present modification, it is possible to provide an imaging device capable of obtaining a high-quality image with high light utilization efficiency.

### Fourth Embodiment

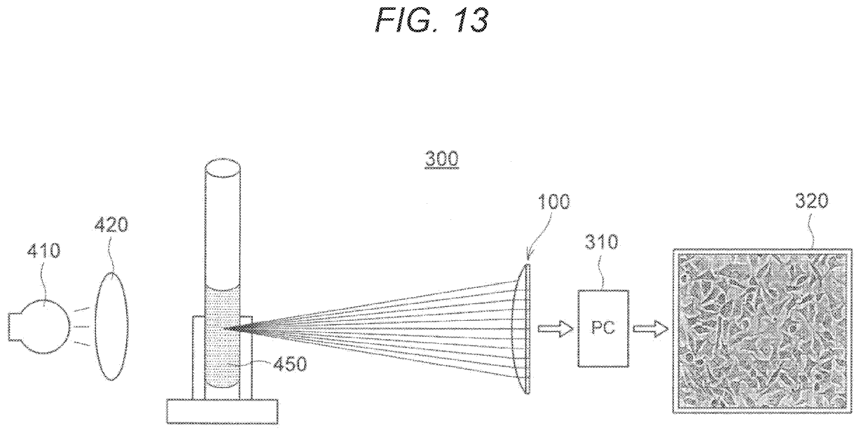

FIG. 13 illustrates an electronic apparatus according to a fourth embodiment. An electronic apparatus 300 is a microscope for observing a nearly transparent subject, such as a cell, and a system for displaying an image thereof, and includes an imaging device 100, a processing unit 310, and a display unit 320. The imaging device 100 is the imaging device according to the third embodiment. A personal computer (PC), for example, is used as the processing unit 310, and processes a signal supplied from the signal processing unit 258 illustrated in FIG. 4 to obtain image data. A display device 320 displays an image by using the image data supplied from the processing unit 310.

In the fourth embodiment, a light beam emitted from a light source 410 irradiates, through a lens 420, a well 450 containing a sample (a cell, for example). The light beam transmitted through the well 450 is imaged by the imaging device 100, an imaging result is processed in the processing unit 310, and a processing result is displayed on the display device 320.

Note that the imaging device according to the first modification or second modification of the third embodiment may be used as the imaging device 100.

As described above, with the electronic apparatus according to the fourth embodiment, a nearly transparent subject, such as a cell, can be observed, and an image obtained has high image quality.

Furthermore, if the imaging device according to the second embodiment and the modifications thereof are used as the imaging device 100 according to the present embodiment, it is possible to obtain an image in which a nearly transparent subject is subjected to color reproduction and color enhancement.

With this arrangement, according to the fourth embodiment, it is possible to obtain an electronic apparatus capable of obtaining a high-quality color image with high light utilization efficiency.

### Fifth Embodiment

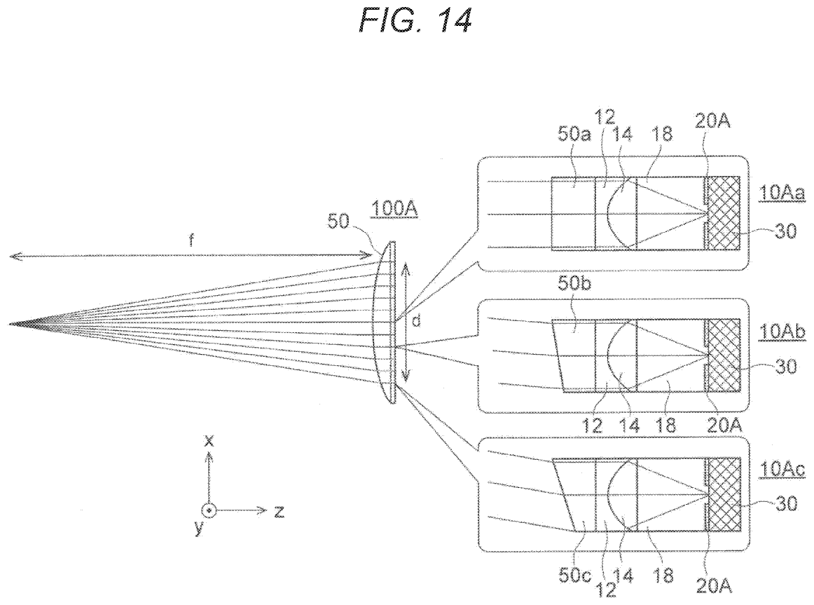

FIG. 14 illustrates an imaging device according to a fifth embodiment. An imaging device 100A according to the fifth embodiment has a configuration of the imaging device 100 according to the third embodiment illustrated in FIG. 10, in which the phase modulation unit 16 is deleted from each pixel, and the light-shielding unit 20 is replaced by a light-shielding unit 20A.

Pixels 10Aa, 10Ab, and 10Ac illustrated in FIG. 14 correspond to the pixels 10a, 10b, and 10c illustrated in FIG. 10, respectively.

The light-shielding unit 20A has a configuration in which the pinhole 20c is deleted from the light-shielding unit 20 illustrated in FIG. 1. That is, in the light-shielding unit 20A, a pinhole 20c provided on a portion of a side surface of a light guide unit 18 is deleted.

In the imaging device 100A configured as described above, similarly to the first embodiment, light is condensed into a pinhole of the light-shielding unit 20A by using a lens unit 14 including a microlens, and therefore light utilization efficiency can be increased.

Note that, similarly to the first embodiment, by newly providing a part 20b that covers the portion of the side surface of the light guide unit 18, crosstalk can be reduced, and light utilization efficiency can be further increased.

### Sixth Embodiment

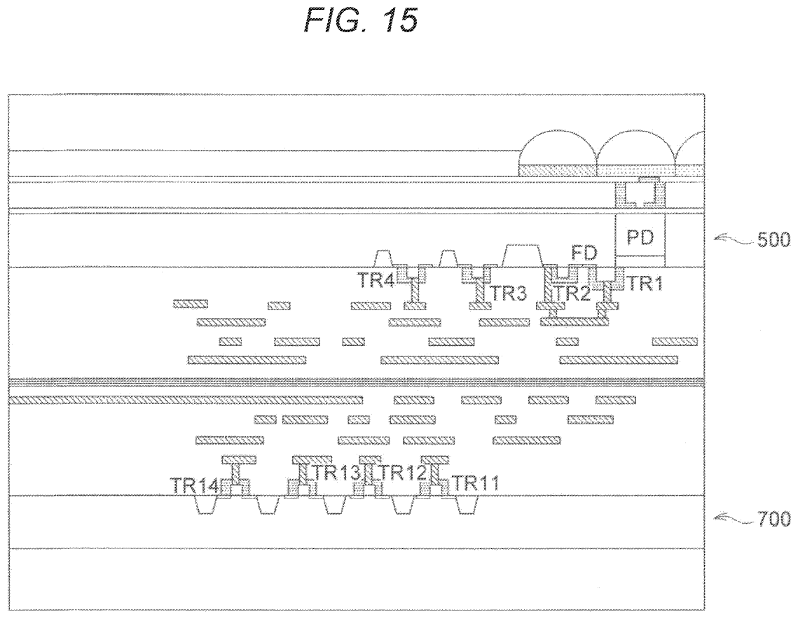

A method for manufacturing an imaging device according to a sixth embodiment will be described with reference to FIGS. 15 to 23. The manufacturing method according to the sixth embodiment manufactures a back-illuminated MOS imaging device.

FIG. 15 is a schematic cross-sectional configuration diagram of an imaging device manufactured with the manufacturing method according to the present embodiment. In the imaging device, a first semiconductor chip unit 500 including a pixel array (hereinafter, also referred to as a pixel region) and a control circuit, and a second semiconductor chip unit 700 equipped with a logic circuit are electrically connected and vertically laminated.

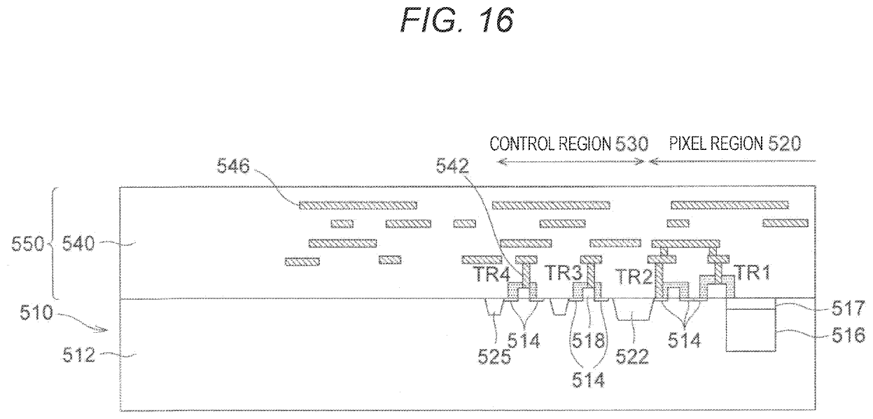

In the sixth embodiment, first, as illustrated in FIG. 16, a semi-manufactured image sensor, that is, a pixel region 520 and control region 530, is formed in a region serving as each chip unit of a first semiconductor wafer (hereinafter, also referred to as a first semiconductor substrate) 510. That is, a photodiode (PD) serving as a photoelectric conversion unit of each pixel is formed in a region serving as each chip unit of the first semiconductor substrate 510 including, for example, a silicon substrate, and a source/drain region 514 of each pixel transistor is formed in the semiconductor well region 512.

The semiconductor well region 512 is formed by introducing an impurity of a first conductivity type, for example, a p-type, and the source/drain region 514 is formed by introducing an impurity of a second conductivity type, for example, an n-type. The photodiode (PD) and the source/drain region 514 of each pixel transistor are formed by ion implantation from a front surface of a substrate. The photodiode (PD) has an n-type semiconductor region 516 and a p-type semiconductor region 517 on a side close to the front surface of the substrate.

A gate electrode 518 is formed on the front surface of the substrate, which constitutes a pixel, via a gate insulation film, and pixel transistors Tr1 and Tr2 are formed with the gate electrode 518 and a pair of source/drain regions 514.

As illustrated in FIG. 16, a plurality of pixel transistors is represented by the two pixel transistors Tr1 and Tr2. The pixel transistor Tr adjacent to the photodiode (PD) corresponds to a transfer transistor, and a source/drain region thereof corresponds to a floating diffusion (FD).

Each unit pixel is separated in an element separation region 522. Meanwhile, in the control region 530, a MOS transistor that constitutes the control region 530 is formed in the first semiconductor substrate 510. FIG. 16 illustrates MOS transistors that constitute the control region 530, as represented by MOS transistors Tr3 and Tr4. Each of the MOS transistors Tr3 and Tr4 includes an n-type source/drain region 514 and a gate electrode 518 formed via a gate insulation film. The MOS transistors Tr3 and Tr4 are separated in an element separation region 525.

Next, a first interlayer insulation film 540 is formed on a front surface of the first semiconductor substrate 510, and then a contact hole is formed in the interlayer insulation film 540 to form a connection conductor 542 connected to a required transistor. When connection conductors 542 having different heights are formed, on an entire surface including an upper surface of a transistor, formed of a silicon-oxide film, for example, as a first thin insulation film (not illustrated), and formed of a silicon-nitride film, for example, as a second thin insulation film (not illustrated) serving as an etching stopper, and laminated. A second interlayer insulation film 540 is formed on the second thin insulation film. Thereafter, contact holes having different depths are selectively formed in the second interlayer insulation film 540 up to a second thin insulation film (not illustrated) serving as an etching stopper.

Next, the first thin insulation film (not illustrated) and the second thin insulation film (not illustrated) having the same film thickness are selectively etched in each unit so as to be connected to each contact hole, to form a contact hole. Then, the connection conductor 542 is embedded in each contact hole.

Next, a plurality of layers, three layers in this example, of copper wiring lines 546 is formed to be connected to each connection conductor 542 via the interlayer insulation film 540, by which. A first multilayer wiring layer 550 is formed. Usually, each copper wiring line 546 is covered with a barrier metal layer (not illustrated) in order to prevent Cu diffusion.

The first multilayer wiring layer 550 is formed by alternately forming an interlayer insulation film 540 and a copper wiring line 546 formed via a barrier metal layer. In the present embodiment, the first multilayer wiring layer 550 is formed with the copper wiring lines 546, but the first multilayer wiring layer 550 may be a metal wiring line including another metal material.

In the processes so far, there is formed the first semiconductor chip unit 500 including the first multilayer wiring layer 550 on an upper part thereof, and including the semi-manufactured pixel region 520 and control region 530.

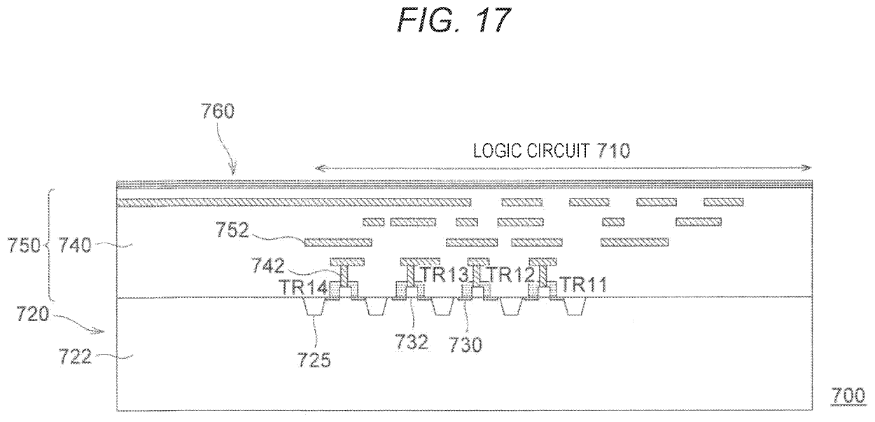

Meanwhile, as illustrated in FIG. 17, a logic circuit 710 including a semi-manufactured signal processing circuit for performing signal processing is formed in a region serving as each chip unit of a second semiconductor substrate (semiconductor wafer) 720 including silicon, for example. That is, a plurality of MOS transistors that constitutes the logic circuit 710 is formed on a p-type semiconductor well region 722 on a front-surface side of the second semiconductor substrate 720, so as to be separated in an element separation region 725. Here, the plurality of MOS transistors is represented by MOS transistors Tr11, Tr12, and Tr13. Each of the MOS transistors Tr1, Tr12, and Tr13 includes a pair of n-type source/drain regions 730 and a gate electrode 732 formed via a gate insulation film (not illustrated). The logic circuit 710 can include a CMOS transistor.

Next, a first interlayer insulation film 740 is formed on a front surface of the second semiconductor substrate 720, and then a contact hole is formed in the interlayer insulation film 740. A connection conductor 742 connected to a required transistor is formed, so as to be embedded in the contact hole. When connection conductors 742 having different heights are formed, as in a case described above, on an entire surface including an upper surface of a transistor, there are laminated a silicon-oxide film, for example, as a first thin insulation film (not illustrated), and a silicon-nitride film, for example, as a second thin insulation film (not illustrated) serving as an etching stopper. A second interlayer insulation film 740 is formed on the second thin insulation film. Then, contact holes having different depths are selectively formed in the second interlayer insulation film 640 up to a second thin insulation film (not illustrated) serving as an etching stopper.

Next, the first thin insulation film (not illustrated) and the second thin insulation film (not illustrated) having the same film thickness are selectively etched in each unit so as to be connected to each contact hole, to form a contact hole.

Then, the connection conductor 742 is embedded in each contact hole. Thereafter, formation of the interlayer insulation film 740 and formation of the plurality of layers of metal wiring lines are repeated to form a second multilayer wiring layer 750. In the present embodiment, four layers of copper wiring lines 752 are formed by using a process similar to the process of forming the first multilayer wiring layer 550 formed on the first semiconductor substrate 510, and the second multilayer wiring layer 750 is formed.

Then, a warpage correction film 760 for reducing warpage when a first semiconductor substrate 610 and the second semiconductor substrate 720 are bonded together is formed on an upper part of the second multilayer wiring layer 750. In the processes so far, there is formed the second semiconductor substrate 720 including the second multilayer wiring layer 750 on an upper part thereof, and including the semi-manufactured logic circuit.

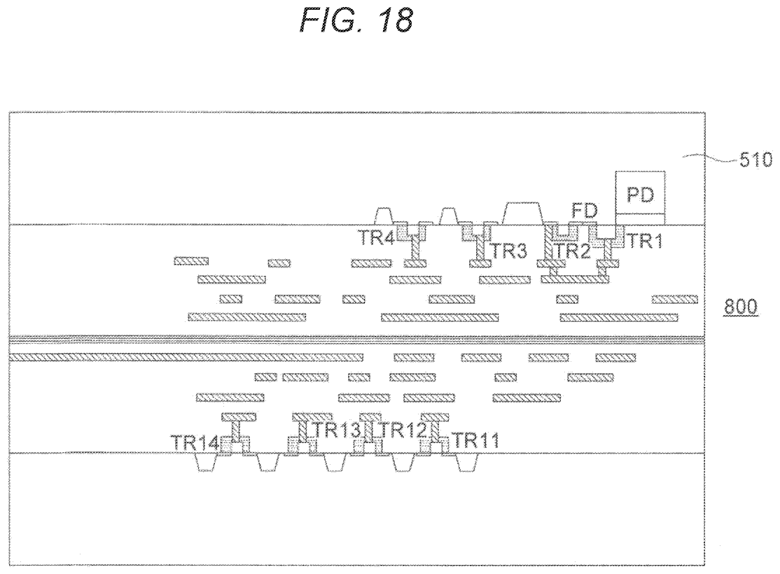

Next, as illustrated in FIG. 18, the first semiconductor substrate 510 and the second semiconductor substrate 720 are bonded together such that the first multilayer wiring layer 550 and the second multilayer wiring layer 750 face each other. The bonding is performed with, for example, an adhesive. Alternatively, the bonding may be performed with plasma bonding. Then, the first multilayer wiring layer 550 having a multilayer wiring layer on an upper part thereof and the second semiconductor substrate 720 are laminated and bonded to each other, thereby forming a laminated body 800 including two dissimilar substrates.

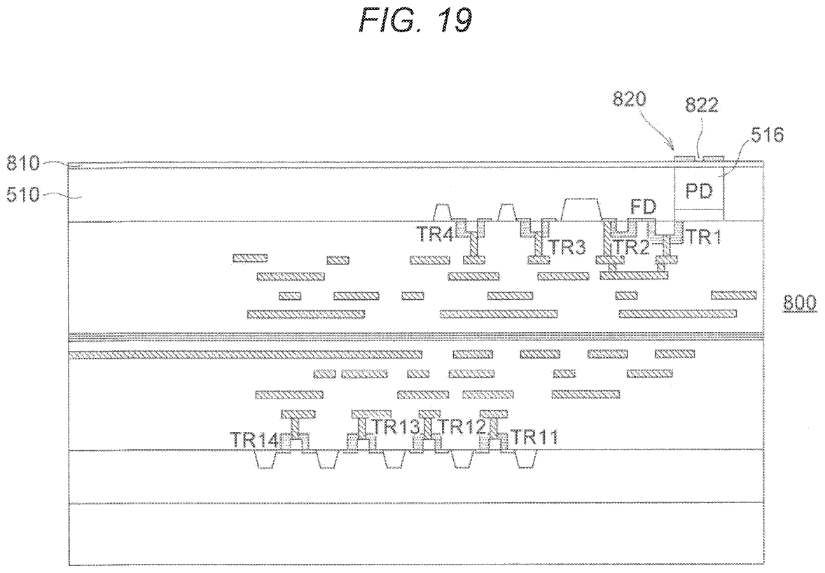

Next, as illustrated in FIG. 19, the first semiconductor substrate 510 is ground and polished from a back surface side thereof to be thinned. This thinning is performed such that a back surface of the photodiode (PD), that is, the n-type semiconductor region 516, is exposed. After the first semiconductor substrate 510 is thinned, a p-type semiconductor layer (not illustrated) for reducing dark current is formed on the back surface of the photodiode (PD). A thickness of the first semiconductor substrate 510 is, for example, about 600 m, but the first semiconductor substrate 510 is thinned to, for example, about 3 m to 5 m. The back surface of the first semiconductor substrate 510 serves as a light incident surface when configured as a back-illuminated imaging device.

Next, an antireflection coating 810 is applied to the back surface of the first semiconductor substrate 510. Subsequently, as illustrated in FIG. 19, a tungsten film 820 having a thickness of, for example, 350 nm is formed on the photodiode (PD). Thereafter, a front surface is polished by a chemical mechanical polishing (CMP) method, and a pinhole 822 is formed with etching.

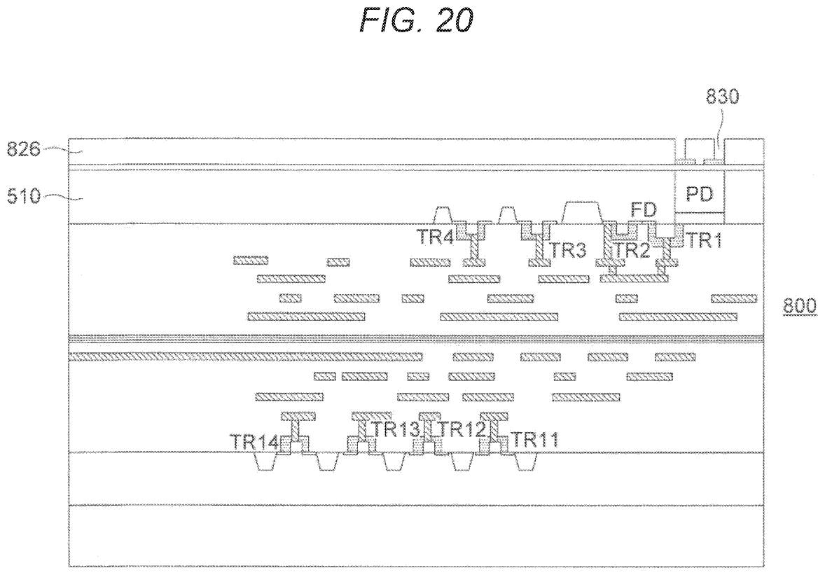

Next, as illustrated in FIG. 20, a light-shielding film groove part 830 that requires light shielding is formed. The light-shielding film groove part 830 is formed by forming an opening with etching from an upper surface of an insulation film 826 formed on the back surface side of the first semiconductor substrate 510. The opening is formed at a depth not reaching the first semiconductor substrate 510, for example.

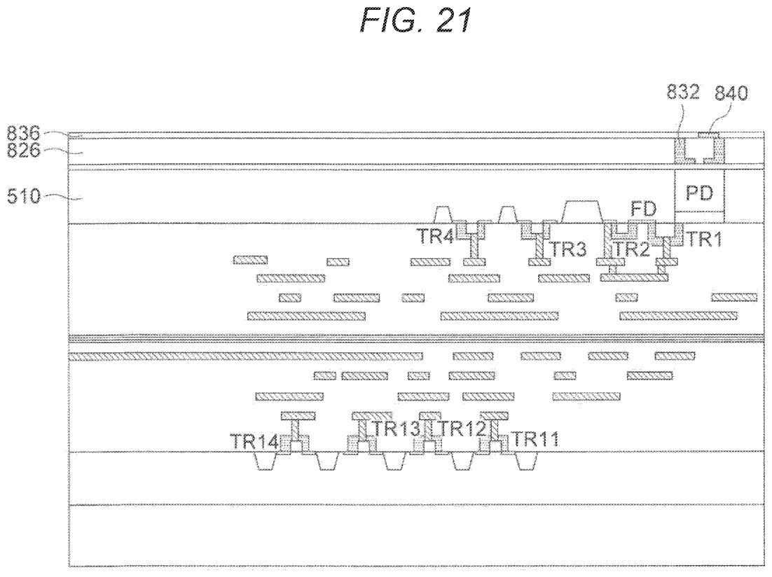

Thereafter, as illustrated in FIG. 21, for example, a tungsten (W) film is formed, and a front surface thereof is polished by the CMP method. With this arrangement, tungsten in the light-shielding film groove part 830 is left, and a light-shielding film 832 is formed in a light-shielding region.

Thereafter, a planarization film 836 is formed on an entire surface. Subsequently, a ½-wave phase plate 840 that occupies an area of about ½ of a sensor effective diameter is formed.

The phase plate 840 forms a thin film of silicon nitride on the planarization film 836 for example, and forms a photoresist on the silicon-nitride film. A mask (not illustrated) having a hole in a shape of the ½-wave phase plate is formed on the photoresist. Subsequently, exposure and development are performed, and then the photoresist in a region not covered with the above-described mask is removed. Then, by using the mask described above. The silicon-nitride film is etched, then the photoresist is peeled off, and cleaning is performed, by which forming can be performed. Note that the above-described mask is removed when the photoresist is peeled off.

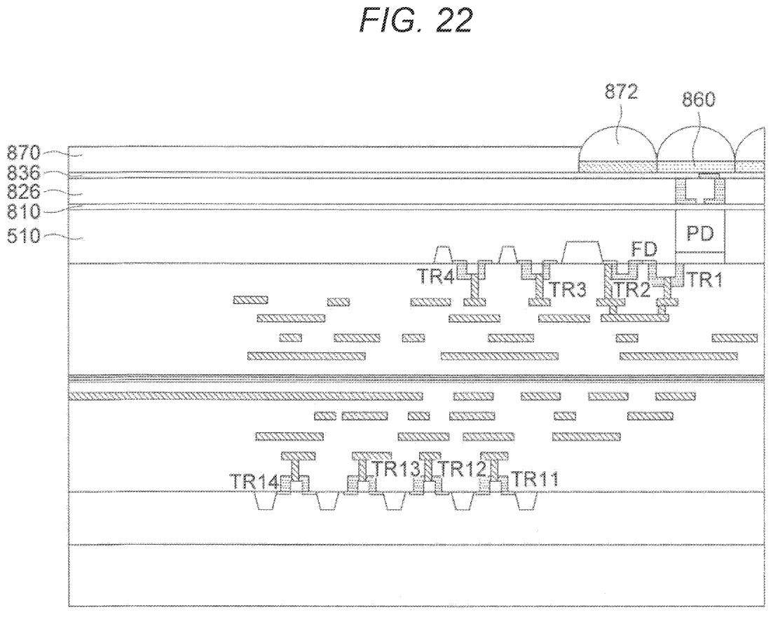

Next, as illustrated in FIG. 22, for example, on-chip color filters 860 of red (R), green (G), and blue (B) are formed corresponding to respective pixels on the planarization film 836. The on-chip color filters 860 can be formed on an upper part of the photodiode (PD) that constitute a desired pixel array, by forming and patterning an organic film containing a pigment or dye of a desired color. Thereafter, an on-chip lens material 870 is formed in a pixel array region including an upper part of the on-chip color filters 860.

Next, as illustrated in FIG. 22, a resist film for on-chip lens is formed in a region corresponding to each pixel on an upper part of the on-chip lens material 870, and etching processing is performed to form an on-chip lens 872.

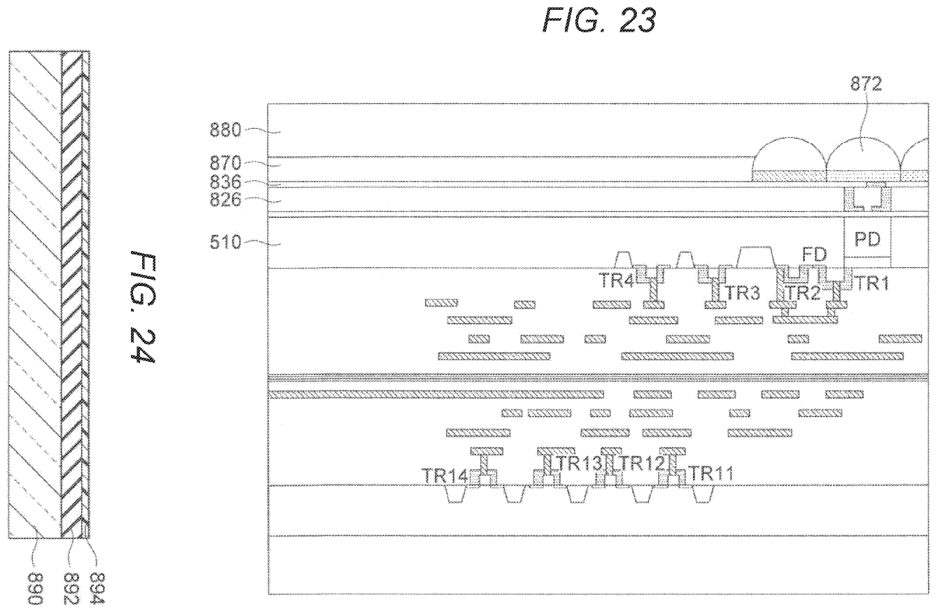

The convex lens 50 according to the third embodiment of the present disclosure illustrated in, for example, FIG. 10 is formed on the on-chip lens 872 with an air gap provided. Furthermore, as illustrated in FIG. 23, an adhesive layer 880 may be formed to form the convex lens 50. Furthermore, the Fresnel lens 52 illustrated in FIG. 11 may be formed instead of the convex lens 50. Furthermore, the hologram 54 illustrated in FIGS. 12A and 12B may be formed instead of the convex lens 50. At this time, the convex lens 50 can be bonded by using a plastic molded lens or a glass molded lens. Furthermore, a wafer-level lens manufactured by using a replica process may be formed with step & repeat.

Alternatively, as illustrated in FIG. 23, after the adhesive layer 880 is formed, a cover glass may be bonded, a thickness thereof may be reduced to, for example, 50 m, and then the hologram 54 may be formed.

### Method for Manufacturing Hologram

Next, a method for manufacturing the hologram 54 will be described with reference to FIGS. 24 to 38. The hologram manufactured by the manufacturing method has a four-tier step shape.

As illustrated in FIG. 24, the adhesive layer 880 illustrated in FIG. 23 is formed on an upper layer portion of a light incident surface (back surface), and then a cover glass 890 is bonded. Subsequently, for example, the cover glass 890 is thinned to 50 μm, and a silicon-oxide film 892 having a thickness of 0.4 m is formed on the cover glass 890. A photoresist film 894 is formed on the silicon-oxide film 892.

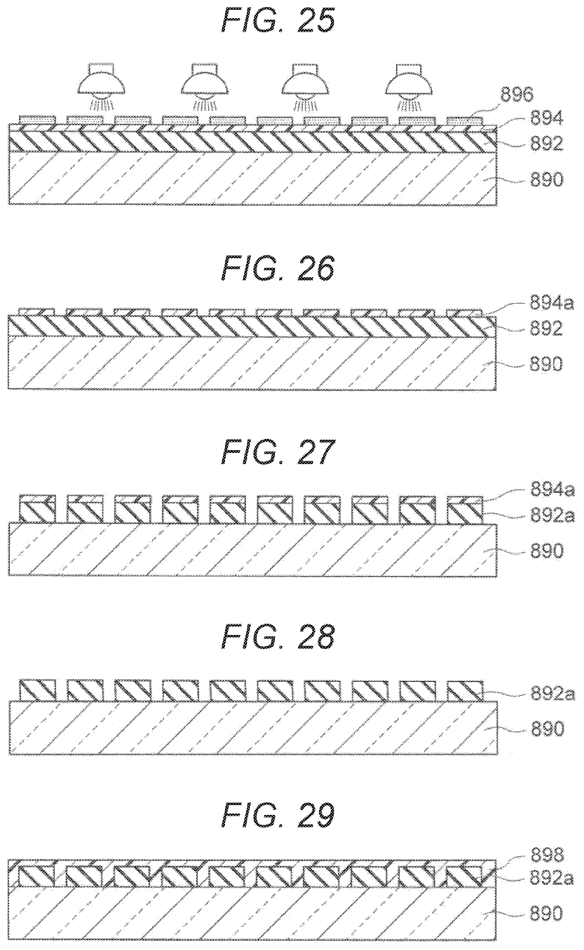

Next, a mask having a hole in a shape of a first tier of the hologram is formed on the photoresist film 894, and the photoresist film 894 is exposed (FIG. 25).

Next, after the development, the exposed photoresist and a mask 896 are removed. With this arrangement, a mask 894a including a photoresist is formed (FIG. 26). Subsequently, the silicon-oxide film 892 is etched by using the mask 894a. With this arrangement, the silicon-oxide film turns into a silicon-oxide film 892a subjected to patterning (FIG. 27). Thereafter, the mask 894a including a photoresist is removed, and then cleaning is performed. With this arrangement, the first tier of a step hologram including the silicon-oxide film 892a is completed (FIG. 28).

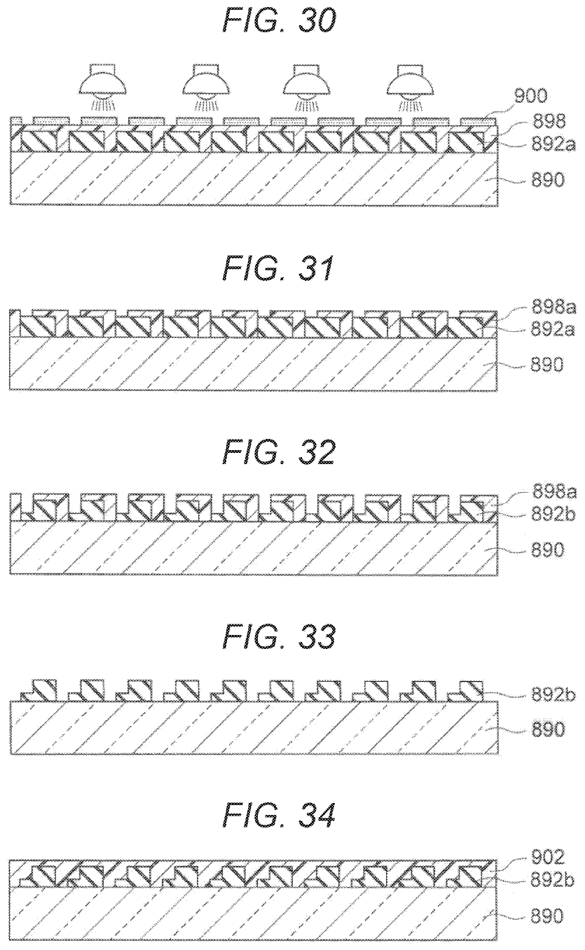

Next, a photoresist film 898 is formed on and between adjacent silicon-oxide films 892a (FIG. 29). Subsequently, a mask 900 provided with a hole in a shape of a second tier of the hologram is formed on the photoresist film 898. The photoresist film 898 is exposed by using the mask 900 (FIG. 30).

Next, the exposed photoresist film 898 is developed, the exposed photoresist film 898 is removed, and the mask 900 is also removed. With this arrangement, a mask 898a including a photoresist is formed (FIG. 31).

Next, the silicon-oxide film 892a is etched by using the mask 898a (FIG. 32). Subsequently, the mask 898a including a photoresist is peeled off, and cleaning is performed. With this arrangement, a silicon-oxide film 892b having the first tier and second tier of the stepped hologram is completed (FIG. 33).

Next, a photoresist film 902 is formed on and between adjacent silicon-oxide films 892b (FIG. 34).

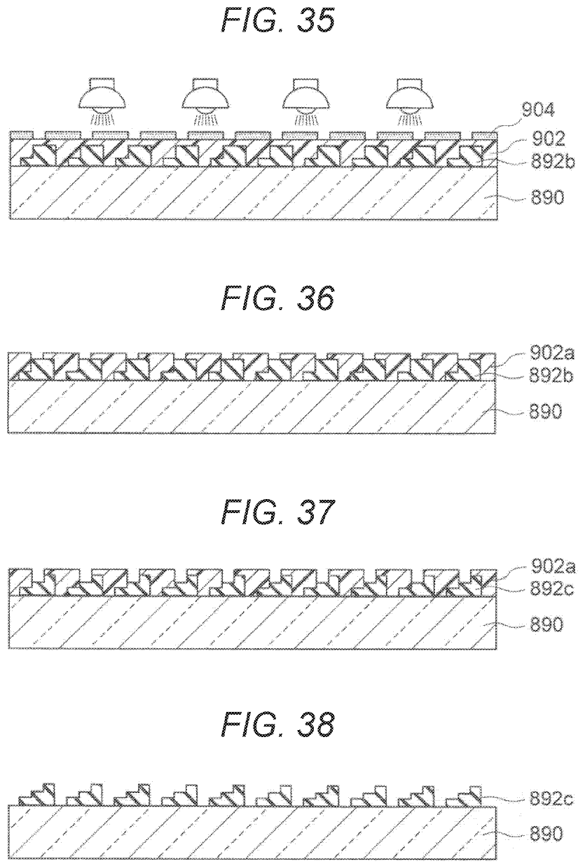

Next, a mask 904 provided with a hole in a shape of a third tier of the hologram is formed on the photoresist film 902. Subsequently, the photoresist film 902 is exposed by using the mask 904 (FIG. 35).

Next, the photoresist film 902 is developed, and the exposed photoresist is removed to form a mask 902a including a photoresist. At this time, the mask 904 is also removed (FIG. 36).

Next, a silicon-oxide film 892b is etched by using the mask 902a. With the etching, the third tier of the stepped hologram is completed, and a silicon-oxide film 892c swinging the first tier, the second cross-section, and the third tier is formed (FIG. 37).

Next, the mask 902a including a photoresist is removed, and cleaning is performed. With this arrangement, a hologram including the silicon-oxide film 892c also having a fourth tier of the stepped hologram is completed (FIG. 38).

The embodiments of the present disclosure have been described above in detail with reference to the accompanying drawings, but the technical scope of the present disclosure is not limited to such examples. It is clear that one of ordinary skill in the technical field of the present disclosure may conceive of various modifications and corrections within the scope of the technical idea recited in claims. It is understood that they also naturally belong to the technical scope of the present disclosure.

Furthermore, the effects described in the present specification are merely exemplary or illustrative, and not restrictive. That is, the technology according to the present disclosure may provide other effects that are apparent to those skilled in the art from the description of the present specification, in addition to or instead of the abovementioned effects.

Note that the following configurations also belong to the technical scope of the present disclosure.

(1) An imaging device including at least one pixel, in which the pixel includes a lens unit that condenses incident light, a phase modulation unit that modulates a phase of some light passed through the lens unit, a light-shielding unit including a pinhole that lets light of which phase is modulated in the phase modulation unit and light of which phase is not modulated pass through, and an imaging element that images light passed through the pinhole.

(2) The imaging device according to (1), in which the phase modulation unit includes a first part and a second part, and is configured to generate a phase difference of ½ of a wavelength between light passed through the first part and light passed through the second part.

(3) The imaging device according to (2), in which, in the phase modulation unit, the first part and the second part have substantially the same area.

(4) The imaging device according to any one of (1) to (3), the imaging device further including a first light guide unit that is disposed closer to a subject than the lens unit is and guides subject light to the lens unit.

(5) The imaging device according to any one of (1) to (4), the imaging device further including a second light guide unit that is disposed between the phase modulation unit and the light-shielding unit, and guides the light passed through the phase modulation unit to the light-shielding unit.

(6) The imaging device according to (5), in which the light-shielding unit includes a first member including the pinhole and disposed in a direction intersecting a direction in which the light passed through the phase modulation unit propagates, and a second member extending from a peripheral edge portion of the first member in a direction of the phase modulation unit, and disposed on a side portion of the second light guide unit. (7) An imaging device including a plurality of pixels arranged in a matrix, in which each of the pixels includes a lens unit that condenses incident light, a light-shielding unit including a pinhole that lets at least a portion of light passed through the lens unit pass through, and an imaging element that images light passed through the pinhole, at least one pixel of the plurality of pixels includes a phase modulation unit that modulates a phase of the some light passed through the lens unit, and the light-shielding unit of the pixel including the phase modulation unit includes the pinhole that lets light of which phase is modulated in the phase modulation unit and light of which phase is not modulated pass through.

(8) The imaging device according to (7), in which the phase modulation unit includes a first part and a second part, and is configured to generate a phase difference of ½ of a wavelength between light passed through the first part and light passed through the second part.

(9) The imaging device according to (7) or (8), in which, in the phase modulation unit, the first part and the second part have substantially the same area.

(10) The imaging device according to any one of (7) to (9), the imaging device further including a first light guide unit that is disposed closer to a subject than the lens unit is and guides subject light to the lens unit, and a second light guide unit that is disposed between the lens unit and the light-shielding unit, and guides a light beam from the lens unit to the light-shielding unit.

(11) The imaging device according to (10), in which the light-shielding unit extends, with a first member including the pinhole and disposed in a direction intersecting a direction in which the light passed through the phase modulation unit propagates, from a peripheral edge portion of the first member in a direction of the phase modulation unit, and is disposed on a side portion of the second light guide unit.

(12) The imaging device according to any one of (7) to (11), in which the plurality of pixels is disposed in units of a pixel group including first to fourth pixels disposed in two rows and two columns adjacent to each other, the first to third pixels include the phase modulation unit, and the fourth pixel does not include the phase modulation unit, and includes a color filter disposed between the imaging element and the lens unit.

(13) The imaging device according to any one of (7) to (11), in which the plurality of pixels is disposed in units of a pixel group including first to fourth pixels disposed in two rows and two columns adjacent to each other, the first to second pixels include the phase modulation unit, and the third to fourth pixels do not include the phase modulation unit, and include different color filters disposed between the imaging element and the lens unit.

(14) The imaging device according to any one of (7) to (11), in which the plurality of pixels is disposed in units of a pixel group including first to fourth pixels disposed in two rows and two columns adjacent to each other, the first pixel includes the phase modulation unit, and the second to fourth pixels do not include the phase modulation unit, and include color filters different from one another, the color filters being disposed between the imaging element and the lens unit.

(15) The imaging device according to any one of (7) to (11), in which the plurality of pixels is disposed in units of a pixel group including first to fourth pixels disposed in two rows and two columns adjacent to each other, the first pixel includes the phase modulation unit, the second to third pixels do not include the phase modulation unit, and include first color filters of same color, the first color filters being disposed between the imaging element and the lens unit, and the fourth pixel does not include the phase modulation unit, and includes a second color filter of a color different from the color of the first filters, the second color filter being disposed between the imaging element and the lens unit.

(16) The imaging device according to any one of (7) to (15), the imaging device further including an optical member that is disposed between the plurality of pixels and a subject, and condenses light from the subject on the plurality of pixels.

(17) The imaging device according to (16), in which the optical member is a convex lens.

(18) The imaging device according to (16), in which the optical member is a Fresnel lens.

(19) The imaging device according to (16), in which the optical member is a hologram.

(20) An electronic apparatus including an imaging device, and a signal processing unit that performs signal processing on the basis of a pixel signal imaged in the imaging device, in which the imaging device includes a plurality of pixels arranged in a matrix, each of the pixels includes a lens unit that condenses incident light, a light-shielding unit including a pinhole that lets at least a portion of light passed through the lens group pass through, and an imaging element that images light passed through the pinhole, at least one pixel of the plurality of pixels includes a phase modulation unit that modulates a phase of the some light passed through the lens unit, and the light-shielding unit of the pixel including the phase modulation unit includes the pinhole that lets light of which phase is modulated in the phase modulation unit and light of which phase is not modulated pass through.

### REFERENCE SIGNS LIST

10, 10₁₁ to 10₄₄, 10a, 10b, 10c, 10Aa, 10Ab, 10Ac Pixel
12 Light guide unit
14 Lens unit
16 Phase modulation unit
16a, 16b Part
18 Light guide unit
20, 20A Light-shielding unit
20a, 20b Part
20c Pinhole
30 Imaging element
40 Subject
42a, 42b Light beam
44a, 44b Light beam
46a, 46b Light beam
50 Lens
50a, 50b, 50c Region
52 Fresnel lens
54 Hologram
100, 100A Imaging device
250 Imaging device
251 Pixel region (pixel array)
252 Pixel drive line
253 Vertical signal line
254 Vertical drive unit
255 Column processing unit
256 Horizontal drive unit
257 System control unit
258 Signal processing unit
259 Memory unit
300 Electronic apparatus
310 Processing unit
320 Display unit
410 Light source
420 Lens
450 Well
500 First semiconductor chip unit
510 First semiconductor substrate
512 Semiconductor well region
514 Source/drain region
516 N-type semiconductor region
517 P-type semiconductor region
518 Gate electrode
520 Pixel region
522 Element separation region
525 Element separation region
530 Control region
540 Interlayer insulation film
542 Connection conductor
546 Copper wiring line
550 First multilayer wiring layer
700 Second semiconductor chip unit
710 Logic circuit
720 Second semiconductor substrate (semiconductor wafer)
722 P-type semiconductor well region
725 Element separation region
730 Source/drain region
732 Gate electrode
740 Interlayer insulation film
742 Connection conductor
750 Second multilayer wiring layer
760 Warpage correction film
800 Laminated body
810 Antireflection coating
820 Tungsten film
822 Pinhole
826 Insulation film
830 Light-shielding film groove part
832 Light-shielding film
836 Planarization film
860 On-chip color filter
870 On-chip lens material
872 On-chip lens
880 Adhesive layer
890 Cover glass
892, 892a, 892b, 892c Silicon-oxide film
894 Photoresist film
894a Mask including photoresist
896 Mask
898 Photoresist film
898a Mask including photoresist
900 Mask
902 Photoresist film
902a Mask including photoresist
904 Mask

What is claimed is:

1. An imaging device comprising at least one pixel,
wherein the pixel includes
a lens unit that condenses incident light,
a phase modulation unit that modulates a phase of some light passed through the lens unit,
a light-shielding unit including a pinhole that lets light of which phase is modulated in the phase modulation unit and light of which phase is not modulated pass through, and
an imaging element that images light passed through the pinhole.

2. The imaging device according to claim 1, wherein the phase modulation unit includes a first part and a second part, and is configured to generate a phase difference of ½ of a wavelength between light passed through the first part and light passed through the second part.

3. The imaging device according to claim 2, wherein, in the phase modulation unit, the first part and the second part have substantially a same area.

4. The imaging device according to claim 1, the imaging device further comprising a first light guide unit that is disposed closer to a subject than the lens unit is and guides subject light to the lens unit.

5. The imaging device according to claim 1, the imaging device further comprising a second light guide unit that is disposed between the phase modulation unit and the light-shielding unit, and guides the light passed through the phase modulation unit to the light-shielding unit.

6. The imaging device according to claim 5,
wherein the light-shielding unit includes a first member including the pinhole and disposed in a direction intersecting a direction in which the light passed through the phase modulation unit propagates, and a second member extending from a peripheral edge portion of the first member in a direction of the phase modulation unit, and disposed on a side portion of the second light guide unit.

7. An imaging device comprising a plurality of pixels arranged in a matrix,
wherein each of the pixels includes
a lens unit that condenses incident light,
a light-shielding unit including a pinhole that lets at least a portion of light passed through the lens unit pass through, and
an imaging element that images light passed through the pinhole,
at least one pixel of the plurality of pixels includes a phase modulation unit that modulates a phase of the some light passed through the lens unit, and
the light-shielding unit of the pixel including the phase modulation unit includes the pinhole that lets light of which phase is modulated in the phase modulation unit and light of which phase is not modulated pass through.

8. The imaging device according to claim 7, wherein the phase modulation unit includes a first part and a second part, and is configured to generate a phase difference of ½ of a wavelength between light passed through the first part and light passed through the second part.

9. The imaging device according to claim 8, wherein, in the phase modulation unit, the first part and the second part have substantially a same area.

10. The imaging device according to claim 7, the imaging device further comprising:
a first light guide unit that is disposed closer to a subject than the lens unit is and guides subject light to the lens unit; and
a second light guide unit that is disposed between the lens unit and the light-shielding unit, and guides a light beam from the lens unit to the light-shielding unit.

11. The imaging device according to claim 10,
wherein the light-shielding unit includes a first member including the pinhole and disposed in a direction intersecting a direction in which the light passed through the phase modulation unit propagates, and a second member extending from a peripheral edge portion of the first member in a direction of the phase modulation unit, and disposed on a side portion of the second light guide unit.

12. The imaging device according to claim 11,
wherein the plurality of pixels is disposed in units of a pixel group including first to fourth pixels disposed in two rows and two columns adjacent to each other,
the first to third pixels include the phase modulation unit, and
the fourth pixel does not include the phase modulation unit, and includes a color filter disposed between the imaging element and the lens unit.

13. The imaging device according to claim 11,
wherein the plurality of pixels is disposed in units of a pixel group including first to fourth pixels disposed in two rows and two columns adjacent to each other,
the first to second pixels include the phase modulation unit, and
the third to fourth pixels do not include the phase modulation unit, and include different color filters disposed between the imaging element and the lens unit.

14. The imaging device according to claim 11,
wherein the plurality of pixels is disposed in units of a pixel group including first to fourth pixels disposed in two rows and two columns adjacent to each other,
the first pixel includes the phase modulation unit, and
the second to fourth pixels do not include the phase modulation unit, and include color filters different from one another, the color filters being disposed between the imaging element and the lens unit.

15. The imaging device according to claim 11,
wherein the plurality of pixels is disposed in units of a pixel group including first to fourth pixels disposed in two rows and two columns adjacent to each other,
the first pixel includes the phase modulation unit,
the second to third pixels do not include the phase modulation unit, and include first color filters of same color, the first color filters being disposed between the imaging element and the lens unit, and
the fourth pixel does not include the phase modulation unit, and includes a second color filter of a color different from the color of the first filters, the second color filter being disposed between the imaging element and the lens unit.

16. The imaging device according to claim 7, the imaging device further comprising an optical member that is disposed between the plurality of pixels and a subject, and condenses light from the subject on the plurality of pixels.

17. The imaging device according to claim 16, wherein the optical member is a convex lens.

18. The imaging device according to claim 16, wherein the optical member is a Fresnel lens.

19. The imaging device according to claim 16, wherein the optical member is a hologram.

20. An electronic apparatus comprising:
an imaging device; and
a signal processing unit that performs signal processing on a basis of a pixel signal imaged in the imaging device,
wherein the imaging device includes a plurality of pixels arranged in a matrix,
each of the pixels includes
a lens unit that condenses incident light,
a light-shielding unit including a pinhole that lets at least a portion of light passed through the lens unit pass through, and
an imaging element that images light passed through the pinhole,
at least one pixel of the plurality of pixels includes a phase modulation unit that modulates a phase of the some light passed through the lens unit, and
the light-shielding unit of the pixel including the phase modulation unit includes the pinhole that lets light of which phase is modulated in the phase modulation unit and light of which phase is not modulated pass through.
